(19) United States
(12) Patent Application Publication
(10) Pub. No.: US 2009/0220930 A1
(43) Pub. Date: Sep. 3, 2009

(54) Time Varying Electromagnetic Force Sleeve for the Expansion of Cells and Method of Using the Same

(76) Inventor: Clayton R. Parker, Safety Harbor, FL (US)

Correspondence Address: LADAS & PARRY LLP, 224 SOUTH MICHIGAN AVENUE, SUITE 1600, CHICAGO, IL, 60604, US

(21) Appl. No.: 11/993,898

(22) PCT Filed: Jun. 22, 2006
(86) PCT No.: PCT/US2006/024241

§ 371 Date: Nov. 24, 2008

Related U.S. Application Data
(63) Continuation-in-part of application No. 11/169,386, filed on Jun. 29, 2005.
Application No. 11/169,386 is a continuation-in-part of application No. 11/169,613, filed on Jun. 29, 2005.

(51) Int. Cl. A01N 1/02; C12M 3/00

(52) U.S. Cl. 435/2; 435/286.1

(57) ABSTRACT

A time varying electromagnetic force sleeve wherein the time varying electromagnetic force sleeve comprises a time varying electromagnetic force source operatively connected to an electrically conductive coil that can removably receive a culture container. The present invention also relates to a method for cell expansion comprising providing a time varying electromagnetic force sleeve that is introduced to a culture container and that in use delivers a time varying electromagnetic force to cells contained within the culture container for cell expansion.

### FIELD OF THE INVENTION

The present invention relates generally to a sleeve for the expansion of cells, and more particularly to a time varying electromagnetic force sleeve that removably recieves a culture container. The present invention also relates to a time varying electromagnetic force sleeve that, when in use, supplies a time varying electromagnetic force to cells in a culture container for cell expansion therein.

### BACKGROUND OF THE INVENTION

For many years there have existed methods for culturing cells. Some methods involve culturing cells in two-dimensional cultures utilizing such culture chambers as flasks and petri dishes, and others involve culturing cells in three-dimensional cultures utilizing such culture chambers as bioreactors. Methods of optimally culturing cells include adding molecules to cells in a culture such as growth factors, hormones, and others that, for instance, up or down regulate expansion of cells. Some methods are optimized for culturing individual cells and others are optimized for tissue culture. In addition, in an effort to produce more cells or larger tissue constructs over time, cell cultures are performed under various conditions.

U.S. Pat. No. 6,673,597, Wolf et al., disclose the use of a time varying electromagnetic force (“TVEMF”) to grow cells in culture. Wolf et al., disclose the use of electrodes to supply an electromagnetic force to a cell culture for growth of the same. In those instances where a conductive coil, rather than electrodes, is used for the delivery of a TVEMF to a cell culture, the coil is integral with a culture chamber and affixed thereto. Since the cells expanded in a culture may be introduced into the human body for tissue regeneration or treatment of human maladies, the culture chamber should be sterile. If the source of TVEMF to be delivered to cells in a culture chamber is integral with the culture chamber then either the culture chamber should be sterilized without disturbing the source of the TVEMF, a process which is cumbersome and time consuming, and if the container is to be discarded after each use, then the integral TVEMF coil has to be discarded therewith. Either way, an integral TVEMF coil is an expensive approach to cell expansion.

Consequently, it would be highly desirable to have a TVEMF sleeve comprising an electrically conductive coil having an interior portion wherein the interior portion defines a space wherein a culture container is removably received. It is also highly desirable to have a TVEMF sleeve comprising a coil support with an interior portion and an exterior portion, an electrically conductive coil wrapped around the exterior portion of the coil support, and a TVEMF source, and wherein the interior portion of the coil support defines a space wherein a culture container is removably received. It is further highly desirable to have a TVEMF sleeve that, when in use, is introduced to a culture container containing cells and supplies a TVEMF to the cells in a culture container for cell expansion. The present invention overcomes the problems associated with past and current cell culture methods, and presents advantages not before seen.

### SUMMARY OF THE INVENTION

The present invention relates to a TVEMF sleeve comprising an electrically conductive coil having an interior portion and an exterior portion wherein the interior portion defines a space wherein a culture container is removably received, and a TVEMF source operatively connected to the electrically conductive coil. Preferably the electrically conductive coil is substantially rigid.

The present invention also relates to a TVEMF sleeve comprising a coil support with an interior portion and an exterior portion wherein the interior portion defines a space in which a culture container is removably received, an electrically conductive coil wrapped around the exterior portion of the coil support, and a TVEMF source operatively connected to the electrically conductive coil.

The present invention also relates to a method of cell expansion comprising the steps of providing a time varying electromagnetic force sleeve comprising an electrically conductive coil, having an interior portion and exterior portion, operatively connected to a time varying electromagnetic force source; introducing a culture container containing cells to the interior portion of the electrically conductive coil of the time varying electromagnetic force sleeve; and supplying a time varying electromagnetic force to the cells in the culture container through the time varying electromagnetic force sleeve for cell expansion. The TVEMF sleeve may preferably deliver a TVEMF in the form of a pulsed square wave (following a Fourier curve) to the cells in the culture container, and the TVEMF may preferably be of from about 0.05 gauss to about 6 gauss.

Other aspects, features, and advantages of the present invention will be apparent from the following description of the preferred embodiments of the invention given for the purpose of disclosure. This invention may be more fully described by the preferred embodiment(s) as hereinafter described, but is not intended to be limited thereto.

### BRIEF DESCRIPTION OF THE DRAWINGS

In the drawings,

FIG. 1 is an elevated side view of a TVEMF sleeve;

FIG. 2 is an elevated front view of a TVEMF sleeve;

FIG. 3 is an elevated front view of a TVEMF sleeve removably adjacent to and encompassing a culture container;

FIG. 4 is an elevated side view of a TVEMF sleeve;

FIG. 5 is an elevated side view of a TVEMF sleeve being introduced to a culture container;

FIG. 6 is an elevated side view of a TVEMF sleeve;

FIG. 7 is a cross sectional elevated side view of a TVEMF sleeve being introduced to a rotatable culture container; and

FIG. 8 is a cross sectional elevated side view of a TVEMF sleeve removably adjacent to and encompassing a rotatable culture container.

### DETAILED DESCRIPTION OF THE DRAWINGS

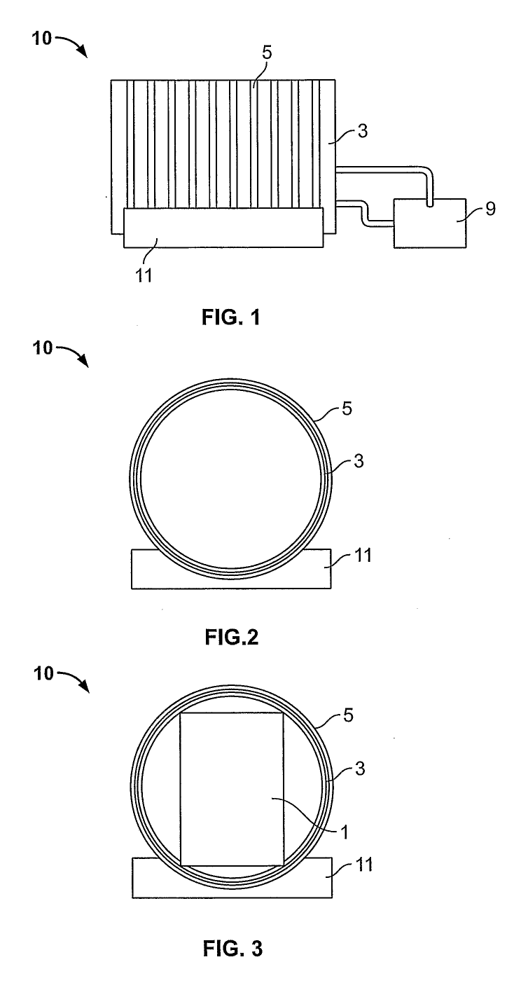

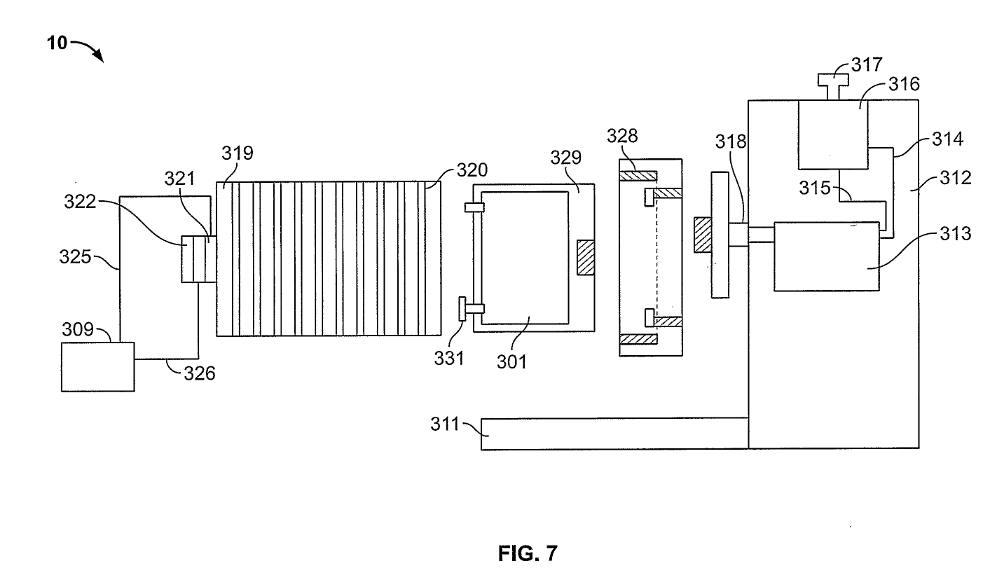

In the drawings, FIGS. 1-6 illustrate preferred embodiments of a TVEMF sleeve 10 that may supply a TVEMF to cells inside a culture container. FIGS. 7 and 8 illustrate a preferred embodiment of a TVEMF sleeve 10 that is rotatable and that may supply a TVEMF to cells inside a rotatable culture container.

FIG. 1 is an elevated side view of a preferred embodiment of the TVEMF sleeve 10 comprising an electrically conductive coil 5 and a TVEMF source 9 operatively connected to the electrically conductive coil 5. The phrase “operatively connected,” and similar words and phrases, is intended to mean that the TVEMF source can be connected to the electrically conductive coil in a manner such that when in operation, the TVEMF source can impart a TVEMF to the electrically conductive coil through a conductive connection, preferably with at least one electrically conductive wire. The TVEMF source may preferably be affixed to the electrically conductive coil, and may preferably be integral with the electrically conductive coil. In the present invention, the electrically conductive coil may be any material that conducts electricity. The electrically conductive coil may preferably be, but is not limited to, the following conductive materials; silver, gold, copper, aluminum, iron, lead, titanium, uranium, a ferromagnetic metal, and zinc, or a combination thereof. The electrically conductive coil may also preferably comprise salt water. The electrically conductive coil of the present invention may preferably be substantially rigid. “Substantially rigid,” is intended throughout this invention to mean that the electrically conductive coil can maintain a shape without the need for support. The electrically conductive coil may be any shape, preferably substantially cylindrical preferably with a substantially elliptical cross-section, more preferably with a substantially oval cross-section, and most preferably with a substantially circular cross-section. The electrically conductive coil may also preferably be a solenoid, a tightly wound electrically conductive coil. Furthermore, the electrically conductive coil may preferably be wrapped in electric insulators comprising, but not limited to, rubber, plastic, silicones, glass, and ceramic. Preferably the electric insulator provides rigidity to the electrically conductive coil.

In the preferred embodiment illustrated in FIG. 1, the electrically conductive coil 5 is wrapped around the exterior portion of a coil support 3. The coil support of the present invention may preferably be made of a non-conductive non-magnetic material, more preferably non-metallic, and most preferably plastic for example polyethylene. Furthermore, the coil support may preferably comprise a conductive material, preferably iron. Not to be bound by the theory, an iron core is thought to enhance the magnetic field of a solenoid. The exterior portion of the coil support has a shape that is compatible with the electrically conductive coil. By “compatible” it is meant that the coil support lends support to the electrically conductive coil and preferably dictates and maintains the shape of the electrically conductive coil. The electrically conductive coil may be wound around the exterior portion of the coil support supporting a shape of the electrically conductive coil, preferably having a substantially oval cross-section, more preferably a substantially elliptical cross-section, and most preferably a substantially circular cross-section. In this preferred embodiment of the TVEMF sleeve 10, the electrically conductive coil 5 and the coil support 3 are disposed on, preferably removably, a base 11 that provides stationary support thereto.

FIG. 2 is an elevated front view of a preferred embodiment of the TVEMF sleeve 10, also illustrated in FIG. 1, depicting the electrically conductive coil 5 wrapped around the exterior portion of the coil support 3 and wherein the electrically conductive coil 5 and the coil support 3 are disposed on, preferably removably, a base 11.

FIG. 3 is an elevated front view of a preferred embodiment of the TVEMF sleeve 10, also illustrated in FIGS. 1 and 2, further having a culture container 1 in the interior portion of the coil support 3 wound with the electrically conductive coil 5. In the present invention, the electrically conductive coil, and the coil support when present, has an interior portion that defines a space in which a culture container is removably received. “Removably received” and similar terms refers to a characteristic of a space wherein a culture container is introduced thereto and removed there from as desired. For example, a culture container is removably received by the space in the interior portion of the electrically conductive coil or the coil support when present. The space can accommodate a culture container so that the culture container may be removed and/or dissembled from the space as desired. The space in the interior portion of the coil support may be any shape and preferably has a side on which at least one culture container can be removably disposed.

The culture container of the present invention may be a culture container which can be encompassed by, and contained within, the interior portion of the electrically conductive coil, and coil support where applicable, including, but not limited to, a bioreactor culture container, a rotatable bioreactor culture container, a flask, a plate, a petri dish, and/or a disposable culture container. By “encompasses” it is meant that the electrically conductive coil, and the coil support where applicable, surrounds the culture container. Furthermore, the culture container may be a single culture container or more than one culture container. The culture container of the present invention is also capable of supporting and maintaining a cell culture, preferrably the expansion of a cell, cell aggregate, tissue, or tissue-like structure.

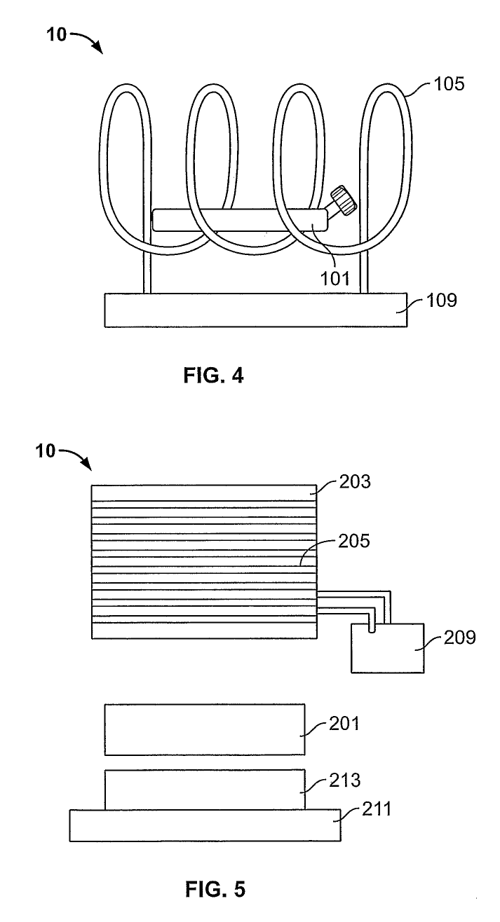

FIG. 4 is an elevated side view of another preferred embodiment of the TVEMF sleeve 10 of the present invention having an electrically conductive coil 105 and an operatively connected TVEMF source 109. The electrically conductive coil 105 is wrapped in a substantially cylindrical shape, preferably with a substantially oval cross-section, and more preferably with a substantially circular cross-section, and most preferably with a substantially elliptical cross-section. The electrically conductive coil 105 in this preferred embodiment is substantially rigid and is preferably insulated. The electric insulator may preferably provide rigidity to the shape of the electrically conductive coil. The electrically conductive coil of the TVEMF sleeve 10 has a cross-section that is large enough to removably receive and encompass a culture container. Furthermore, the electrically conductive coil 105 is removably adjacent to the culture container 101. By “removably adjacent to,” and similar terms, it is meant that the electrically conductive coil can be next to, near to, or touching the culture container, and so that it can be easily removed from, or disassembled from, the culture container.

FIG. 5 illustrates an elevated side view of yet another preferred embodiment of the TVEMF sleeve 10 having an electrically conductive coil 205 and a TVEMF source 209 operatively connected to the electrically conductive coil 205. In this preferred embodiment of the TVEMF sleeve 10, the electrically conductive coil 205 is wrapped around the exterior portion of a coil support 203. FIG. 5 further illustrates the TVEMF sleeve 10 being introduced to a culture container 201, for instance a petri dish. In this preferred embodiment, the culture container 201 is placed on a riser 213, which is in turn disposed on a base 211, preferably removably disposed on, so that the riser 213 may be removed from the base 211 and may be cleaned, sterilized, and/or otherwise maintained as needed. The electrically conductive coil 205 is disposed on, preferably removably, the base 211. To introduce the culture container 201 to the TVEMF sleeve 10, the culture container 201 is placed on the riser 213, and the electrically conductive coil 205 and coil support 203 are then placed over the culture container 201. The term “introducing,” and similar terms, is intended to refer to the removable reception and accommodation of a culture container in the interior portion of the electrically conductive coil, or the coil support where applicable. Manipulating a TVEMF sleeve so that it encompasses, and is removably adjacent to, a culture container is considered introducing a TVEMF sleeve to a culture container. Furthermore, manipulating a culture container so that it is encompassed by a TVEMF sleeve is also considered introducing a culture container to a TVEMF sleeve. In the present invention, in use, once introduced, the electrically conductive coil of the TVEMF sleeve is removably adjacent to the culture container and at the same time encompasses the culture container.

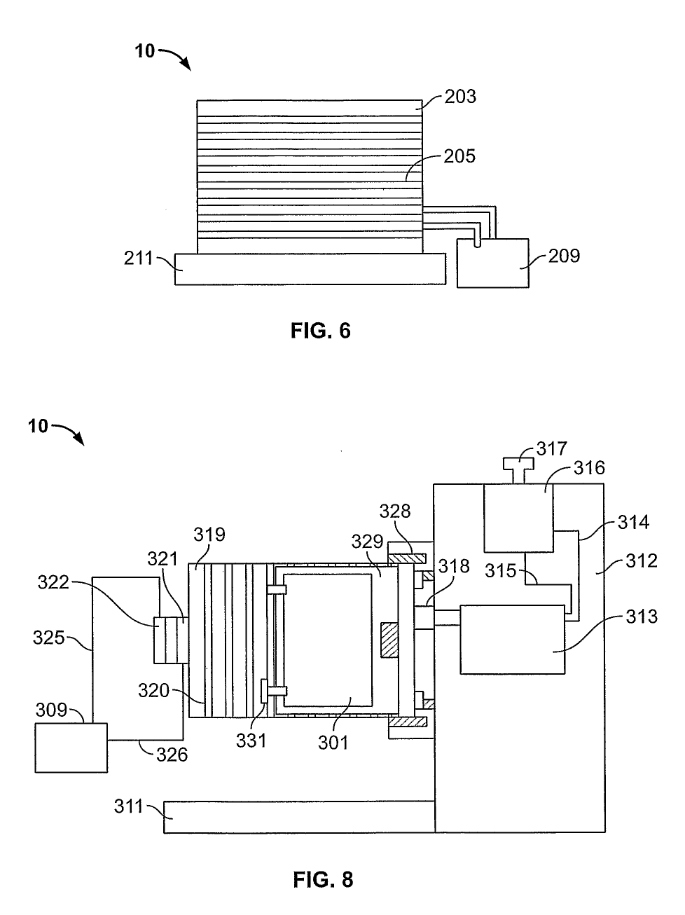

FIG. 6 is an elevated side view of the preferred embodiment of the TVEMF sleeve 10 illustrated in FIG. 5. The TVEMF sleeve 10 comprises an electrically conductive coil 205 and an operatively connected TVEMF source 209. In FIG. 6, as in FIG. 5, the electrically conductive coil 205 is wrapped around the exterior portion of a coil support 203. The coil support 203 is removably adjacent to, and encompasses, a culture container and the coil support 203, wound with the electrically conductive coil 205, is removably disposed on a base 211.

FIG. 7 is a cross sectional elevated side view of another preferred embodiment of a TVEMF sleeve 10 and its introduction to a rotatable culture container 301. The TVEMF sleeve 10 of the preferred embodiment in FIG. 6 comprises an electrically conductive coil 305 and a TVEMF source 309 operatively connected to the electrically conductive coil 305. In this preferred embodiment, the electrically conductive coil 305 is wrapped around the exterior portion of a coil support 303 and the TVEMF sleeve 10 is rotatable. At a first end a first conductive wire 325 and a second conductive wire 326 are connected to the TVEMF source 309. At a second end the wires 325, 326 are connected to at least one ring to facilitate the rotation of the electrically conductive coil 305, preferably a first ring 321 and a second ring 322 respectively. Also in FIG. 7 is depicted a motor housing 312 supported by a base 311. A motor 313 is affixed inside the motor housing 312 and connected by a first wire 314 and a second wire 315 to a control box 316 that houses a control device therein whereby the speed of the motor 313 can be incrementally controlled by turning the control knob 317. Extending from the motor housing 312 is a motor shaft 318. A rotatable mounting 328 removably receives a rotatable culture container holder 329, preferably disposable, that removeably receives a rotatable culture container 301 which is removably affixed within the rotatable culture container holder 329, preferably by a screw 331.

In FIG. 7, preferably the TVEMF sleeve 10 and the rotatable culture container 301 are removably mounted to the rotatable mounting 328. The rotatable mounting 328 is received by the motor shaft 318. When the control knob 317 is turned on, the rotatable culture container 301 along with the coil support 303, wound by the electrically conductive coil 305, may preferably be rotated simultaneously. Furthermore, in operation the TVEMF sleeve 10 remains removably adjacent to, and encompasses, the rotatable culture container 301, while at the same time, supplying a TVEMF to the cells in the rotatable culture container 301. The rotatable culture container may preferably be disposable wherein it can be discarded and a new one used in later cell cultures. The rotatable culture container may also preferably be sterilized, for instance in an autoclave, after each use and re-used for later cell cultures. A disposable culture container could be manufactured and packaged in a sterile environment thereby enabling it to be used by the medical or research professional much the same as other disposable medical devices are used.

FIG. 8 illustrates a cross-sectional elevated side view of the TVEMF sleeve 10 in the preferred embodiment of FIG. 7 removably adjacent to, and encompassing, a rotatable culture container 301.

In operation, a culture container is introduced to a TVEMF sleeve. The TVEMF source of the TVEMF sleeve is turned on and a TVEMF is delivered through the electrically conductive coil into a culture container encompassed by, and removably adjacent to, the TVEMF sleeve. In use, the TVEMF is distributed throughout the culture container, and therefore, to the cells contained therein, preferably in a nearly uniform distribution. In the present invention, the term “cells,” or any other term similar term, is intended to include, but is not limited to single cells, cells attached to cell attachment substrates, cell aggregates, tissues, and tissue-like structures. The term “expansion” is intended to include growth in the size of tissue(s), tissue-like structures, and/or cell aggregates, and/or the growth in the number of cells in a culture container.

Because the present invention provides a method for supplying a TVEMF to cells in a culture container for expansion of the same, a TVEMF sleeve is so sized and configured to removably receive the culture container so that a TVEMF can be supplied to the cells in the culture container, preferably in a nearly uniform distribution. Since the TVEMF sleeve of the present invention is removably adjacent to the culture container, the TVEMF sleeve may be reused for sequential cell cultures. Also, because the TVEMF sleeve is removably adjacent to the culture container, a single TVEMF sleeve can accommodate different types, shapes, and sizes of culture containers.

The size of the electrically conductive coil and the number of times it is wound are such that when a TVEMF is supplied to the electrically conductive coil, a TVEMF is generated in the culture container for the expansion of cells therein. The TVEMF sleeve may generate a TVEMF preferably of from about 0.05 gauss to about 6 gauss, more preferably of from about 0.05 gauss to about 0.5 gauss, and most preferably about 0.5 gauss. The TVEMF is preferably in a delta wave, more preferably in a differentiated square wave, and most preferably in a square wave (following a Fourier curve). Preferably, the pulsed square wave has a frequency of about 2 to about 25 cycles/second, more preferably about 5 to about 20 cycles/second, and for example about 10 cycles/second, and the electrically conductive coil has an RMS value of about 1 to about 1000 mA, preferably about 1 to about 10 mA, for example 6 mA. However, these parameters are not meant to be limiting to the TVEMF of the present invention, and as such, may vary based on other aspects of this invention. TVEMF may be measured by standard equipment, for instance an EN331 Cell Sensor Gauss Meter.

It is further contemplated that the TVEMF sleeve may be equipped with a temperature control device. The temperature control device, preferably be an automated sensor, detects the temperature in the interior portion of the electrically conductive coil, and coil support where applicable. In use, if the temperature control device detects a temperature reading in the interior portion of the electrically conductive coil, and coil support when present, that is higher than desired, the temperature control device may alert the user of the temperature change, preferably with an alarm. Not to be bound by the theory, but when there is a high electrical resistance, heat is generated. Thus, the space wherein a culture container is removably received may become hot depending upon the diameter of the electrically conductive coil and the amount of electricity supplied thereto.

As various changes could be made in TVEMF sleeves, as are contemplated in the present invention, without departing from the scope of the invention, it is intended that all matter contained herein be interpreted as illustrative and not limiting.

### OPERATIVE METHOD

Peripheral blood cells PBCs were collected from donors and a culture mix was made with the whole blood that was collected wherein the cells (0.75×10⁶ cells/ml) were suspended in Iscove's modified Dulbecco's medium (IMDM) (GIBCO, Grand Island, N.Y.) supplemented with 5% human albumin (HA), 100 ng/ml recombinant human G-CSF (Amgen Inc., Thousand Oaks, Calif.), and 100 ng/ml recombinant human stem cell factor (SCF) (Amgen). 10 ppm of D-Penicillamine [D(-)-2-Amino-3-mercapto-3-methylbutanoic acid] (Sigma-Aldrich), a copper chelating agent, dissolved in DMSO, was introduced into the cell mixture. The purpose of the copper chelating agent is, not to be bound by theory, to reduce the amount of copper in the peripheral blood prior to TVEMF-expansion. Not to be bound by theory, it is believed that the decrease in amount of available copper may enhance cell expansion.

### EXAMPLE 1

### Cell Expansion in a Rotatable Bioreactor

A first sample of the culture mix, prepared as above, was placed into a rotatable bioreactor having a 75 ml culture chamber. 0.75×10⁶ cells/ml of the culture mix, was placed in the 75 ml culture chamber, for a total culture mixture volume of 75 ml. A time varying electromagnetic force of about 0.5 gauss in a pulsed square wave was supplied to the cells in the culture container of the rotatable bioreactor through the TVEMF sleeve encompassing and removably adajacent to the rotatable bioreactor, for example in FIGS. 7 and 8. A second sample was placed in a rotatable bioreactor without any TVEMF applied thereto. Other than the TVEMF condition, all other conditions were identical as between the first and second sample. The culture containers were rotated at a speed of about 10 RPM. The cultures were grown at 37° C. and in 5% CO₂.

The cells of the first and second sample were allowed to expand for seven days and after the seventh day of expansion the cells were washed with PBS (Phosphate Buffer Solution) and counted by conventional counting techniques, for example by using a Coulter cell counter. By visual determination, it was found that the first culture mix that was exposed to a TVEMF via the TVEMF sleeve had more than five times the growth or expansion of the sample that was not exposed to a TVEMF.

### EXAMPLE 2

### Cells Expansion in a Petri Dish

A first sample of the culture mix, prepared as above, was placed into a petri dish and a TVEMF of about 0.5 gauss, in a pulsed square wave, was supplied to the TVEMF sleeve that encompassed, and was adjacent to, the petri dish, for example in FIGS. 5 and 6. A second sample was placed in a petri dish without any TVEMF applied thereto. Other than the TVEMF condition, all other conditions were identical as between the first and second sample. The cultures were grown in an incubator at 37° C. and in 5% CO₂.

The cells of the first and second sample were allowed to expand for seven days and after the seventh day of expansion the cells were washed with PBS (Phosphate Buffer Solution) and counted by conventional counting techniques, for example by using a Coulter cell counter. By visual determination it was found that the culture mix that was exposed to a TVEMF via the TVEMF sleeve had more than two times the growth or expansion of the sample that was not exposed to a TVEMF.

1. A time varying electromagnetic force sleeve comprising:
-an electrically conductive coil having an interior portion and an exterior portion wherein the interior portion defines a space in which a rotatable bioreactor is removably received; and
a time varying electromagnetic force source operatively connected to the electrically conductive coil.

2 A time varying electromagnetic force sleeve as in claim 1, wherein the electrically conductive coil is substantially rigid.

3. A time varying electromagnetic force sleeve as in claim 1, wherein the time varying electromagnetic force sleeve is rotatable about an axis.

4. A time varying electromagnetic force sleeve as in claim 3, wherein the axis is substantially horizontal.

5. A time varying electromagnetic force sleeve as in claim 3, wherein the axis is substantially vertical.

6. A time varying electromagnetic force sleeve as in claim 1, wherein the electrically conductive coil is a solenoid.

7. A time varying electromagnetic force sleeve as in claim 1, wherein the electrically conductive coil is substantially cylindrical.

8. A time varying electromagnetic force sleeve as in claim 1, wherein the electrically conductive coil has a substantially circular cross-section.

9. A time varying electromagnetic force sleeve as in claim 1, wherein the electrically conductive coil has a substantially oval cross-section.

10. A time varying electromagnetic force sleeve as in claim 1, wherein the electrically conductive coil has a substantially elliptical cross-section.

11. A time varying electromagnetic force sleeve as in claim 1, wherein the culture container is rotatable about an axis.

12. A time varying electromagnetic force sleeve as in claim 1, wherein the electrically conductive coil is insulated.

13. A time varying electromagnetic force sleeve as in claim 1, further comprising a coil support having an interior portion wherein the coil support is polyethylene, wherein the coil support is located in the interior portion of the electrically conductive coil, and wherein the interior portion of the coil support removably receives the culture container.

14. A time varying electromagnetic force sleeve comprising:
a coil support having an interior portion and an exterior portion wherein the interior portion defines a space in which a rotatable bioreactor is removably received and wherein the coil support is polyethylene;
an electrically conductive coil wound around the exterior portion of the coil support; and
a time varying electromagnetic force source operatively connected to the electrically conductive coil.

15. A time varying electromagnetic force sleeve as in claim 14, wherein the time varying electromagnetic force sleeve is rotatable about an axis.

16. A time varying electromagnetic force sleeve as in claim 15, wherein the axis is substantially horizontal.

17. A time varying electromagnetic force sleeve as in claim 15, wherein the axis is substantially vertical.

18. A time varying electromagnetic force sleeve as in claim 14, wherein the electrically conductive coil is a solenoid.

19. A time varying electromagnetic force sleeve as in claim 14, wherein the electrically conductive coil has a substantially circular cross-section.

20. A time varying electromagnetic force sleeve as in claim 14, wherein the electrically conductive coil has a substantially oval cross-section.

21. A time varying electromagnetic force sleeve as in claim 14, wherein the electrically conductive coil has an elliptical cross-section.

22. A time varying electromagnetic force sleeve as in claim 14, wherein the culture container is rotatable about an axis.

23. A time varying electromagnetic force sleeve as in claim 14, wherein the electrically conductive coil is insulated.

24. A time varying electromagnetic force sleeve as in claim 14, wherein the coil support is non-conductive.

25. A time varying electromagnetic force sleeve as in claim 14, wherein the coil support comprises a conductive material.

26. A time varying electromagnetic force sleeve as in claim 25, wherein the conductive material is iron.

27. A time varying electromagnetic force sleeve as in claim 14, further comprising a temperature control device for controlling the temperature in the culture container.

28. A method of cell expansion comprising the steps of:
providing a time varying electromagnetic force sleeve having an interior portion and exterior portion;
preparing a culture mix comprising peripheral blood cells;
placing the culture mix into a culture container;
introducing the culture container containing peripheral blood cells to the interior portion of the time varying electromagnetic force sleeve;
supplying a time varying electromagnetic force to the cells in the culture container through the time varying electromagnetic force sleeve; and
expanding the peripheral blood cells to a concentration in the culture container within an amount of time.

29. The method of claim 28, wherein the interior portion of the time varying electromagnetic force sleeve further comprises a coil support having an interior portion and an exterior portion wherein an electrically conductive coil is wrapped around the exterior portion of the coil support and wherein the interior portion of the coil support defines a space that removably receives the culture container.

30. The method of claim 28, wherein the interior portion of the time varying electromagnetic force sleeve further comprises an electrically conductive coil having an interior portion and an exterior portion wherein the interior portion defines a space that removably receives the culture container.

31. The method of claim 28 or 29, wherein the time varying electromagnetic force is a square wave.

32. The method of claim 28 or 29, wherein the time varying electromagnetic force is of from about 0.05 gauss to about 6 gauss.

33. The method of claim 28 or 29, wherein the time varying electromagnetic force is of from about 0.05 gauss to about 0.5 gauss.

34. The method of claim 28 or 29, wherein the time varying electromagnetic force is about 0.5 gauss.

35. The method of claim 31, wherein the square wave has a frequency of from about 2 cycles/second to about 25 cycles/second.

36. The method of claim 31, wherein the square wave has a frequency of from about 5 cycles/second to about 20 cycles/second.

37. The method of claim 31, wherein the square wave has a frequency of about 10 cycles/second.

38. The method of claim 28 or 29, wherein the time varying electromagnetic force is a different square wave.

39. The method of claim 28 or 29, wherein the time varying electromagnetic force is a delta wave.

40. The method of claim 28 or 29, wherein the peripheral blood cells that are expanded are peripheral blood adult stem cells.

41. The method of claim 40, wherein the expanding step is for at least seven days.

42. The method of claim 40, wherein the cells are expanded to at least two times the number that were placed into the culture container.

43. The method of claim 40, wherein the cells are expanded to at least five times the number that were placed into the culture container.

44. The method of claim 28 or 29, wherein the culture mix further comprises a copper chelating agent.

45. The method of claim 28 or 29, wherein the culture container is a rotatable bioreactor.

46. The method of claim 45, further comprising the step of rotating the rotatable bioreactor after the step of introducing the rotatable bioreactor to the sleeve.
